(19) United States
(12) Patent Application Publication
(10) Pub. No.: US 2025/0188005 A1
(43) Pub. Date: Jun. 12, 2025

(54) THERMAL COUPLING OF A PLANT FOR PREPARING 1,2-DICHLOROETHANE TO A PLANT FOR THERMAL DESALINATION (OF SEA WATER)

(71) Applicants: thyssenkrupp Uhde GmbH, Dortmund (DE); thyssenkrupp AG, Essen (DE); Westlake Vinnolit GmbH & Co. KG, Ismaning (DE)

(72) Inventors: Michael BENJE, Bad Soden (DE); Peter KAMMERHOFER, Burgkirchen (DE); Klaus KREJCI, Burghausen (DE)

(73) Assignees: thyssenkrupp Uhde GmbH, Dortmund (DE); thyssenkrupp AG, Essen (DE); Westlake Vinnolit GmbH & Co. KG, Ismaning (DE)

(21) Appl. No.: 18/843,142

(22) PCT Filed: Mar. 1, 2023
(86) PCT No.: PCT/DE2023/100163

§ 371 Date: Aug. 30, 2024

(30) Foreign Application Priority Data
Mar. 2, 2022 (DE) 10 2022 104 952.3

(51) Int. Cl. C07C 17/02; B01D 1/00; B01D 3/06; B01D 3/14; B01J 19/00; B01J 19/24; C02F 1/06; C02F 1/16; C02F 103/08

(52) U.S. Cl. CPC C07C 17/02; B01D 1/0058; B01D 3/065; B01D 3/146; B01J 19/0013; B01J 19/2455; C02F 1/06; C02F 1/16; B01J 2219/00087; B01J 2219/00117; C02F 2103/08

(57) ABSTRACT

A method for generation of 1,2-dichloroethane from ethylene and chlorine and for water desalination includes performing the respective processes in plant parts coupled thermally with one another, and allowing heat from the reaction of ethylene with chlorine to be utilized as an energy source for the water desalination. This heat can be utilized extensively by virtue of the coupled water desalination. A corresponding plant is configured for performance of such methods and corresponding methods include using heat from a method for generation of 1,2-dichloroethane from ethylene and chlorine for heating of water to be treated in a water desalination process.

The invention relates to a method for generation of 1,2-dichloroethane from ethylene and chlorine and for water desalination, where the respective processes are performed in plant parts coupled thermally with one another. The present invention further relates to plants configured for performance of such methods and to the use of heat from a method for generation of 1,2-dichloroethane from ethylene and chlorine for heating of water to be treated in a water desalination.

### Prior Art

Polyvinyl chloride (PVC) has a durability, especially in the face of sunlight, that makes it an important polymer for applications in long-lifetime products, in the outdoor segment, for example. Polyvinyl chloride is accordingly produced by polymerization from monomeric vinyl chloride (VCM), which is currently generated normally by way of a two-stage synthesis from ethylene (CH2=CH2) and elemental chlorine (Cl₂), in which first 1,2-dichloroethane (CH2Cl—CH2Cl, also referred to as EDC) is formed as an intermediate. Elimination of hydrogen chloride (HCl) from the 1,2-dichloroethane results subsequently in formation of vinyl chloride:

Cl2+C2H4→C2H4Cl2 (pure EDC)+180 KJ/mol

C2H4Cl2 (cracking EDC)→C2H3Cl (VCM)+HCl−71 KJ/mol

A corresponding process for production of 1,2-dichloroethane from ethylene is described for example in DE 19910964 A1 or DE 102008020386 A1, where the product 1,2-dichloroethane also acts as a reaction medium here. This process approach is also known as direct chlorination. From the reaction equation indicated, it is apparent that the reaction of ethylene with chlorine to 1,2-dichloroethane is strongly exothermic.

In direct chlorination, a distinction is made between low-temperature (LTDC) and high-temperature (HTDC) chlorination processes. The reaction medium in the case of both process variants is the liquid reaction product itself, which contains a dissolved Lewis acid (usually iron (III) chloride) catalyst.

With an LTDC process, described for example in U.S. Pat. No. 2,393,367 A, the reaction is performed at around 50-60° C. The heat of reaction, of around 2000 KJ/kg EDC, is removed entirely by cooling water or else by one or more air coolers. Because of the low reaction temperature, the rate of formation of unwanted byproducts such as 1,1,2-trichloroethane is low as well. However, the low reaction temperature does not permit any heat utilization or heat recovery. Moreover, the reaction product must be washed with water to remove catalyst, in turn producing wastewater that requires separate treatment. Because of these disadvantages, LTDC processes have become much less significant.

With the HTDC processes, the reaction in practice is conducted usually at temperatures between 100° C. and 130° C. The reaction product EDC is taken off from the process in vapor form, with the dissolved catalyst remaining in the reactor. A process of this kind is described for example in GB 1 554 658. With these processes, heat recovery is possible—for example, the reaction product can be fed directly in vapor form into a column for distillative purification or used for indirect heating of distillation columns.

The majority of HTDC processes are conducted as a reactive absorption—that is, chlorine as a gaseous reactant is first dissolved in a circulating stream of the EDC reaction medium, where the circulating EDC stream may come about as a result of forced circulation or natural convection in the reaction setup. In the circulating EDC stream containing dissolved chlorine, gaseous ethylene is then fed in, and after passage through the phase interface reacts with the dissolved chlorine.

It has emerged that, surprisingly, a product of very high quality can be generated even at reaction temperatures of typically 120° C. if the direct chlorination is performed homogeneously in the liquid phase.

The reaction takes place in practice for example in a loop reactor having a riser pipe, a vaporization zone, and a downpipe, in which the reaction mixture is passed in natural convection (i.e., without introduction of flow energy to any substantial extent). The 1,2-dichloroethane produced is taken off in vapor form from the vaporization zone at the upper end of the riser pipe.

The driving force for the natural convection (i.e., the uplift force in the riser pipe) is generated by the addition of the gaseous reactant ethylene in the lower part of the riser pipe on the one hand and by the partial vaporization of the circulating 1,2-dichloroethane in the upper part of the riser pipe on the other hand. The two events lead to a certain gas fraction and hence to a lower mean density in the riser pipe than in the rest of the reactor. The natural convection is supported, moreover, by the temperature of the liquid 1,2-dichloroethane in the riser pipe being higher because of the exothermic reaction and hence by its density being lower by comparison with the 1,2-dichloroethane in the downpipe.

The reaction takes place in liquid phase, by the addition, in gas form via a manifold in the lower part of the riser pipe, of gaseous ethylene which then dissolves in the upward-flowing 1,2-dichloroethane.

In practice, for introducing the chlorine into the 1,2-dichloroethane in the process of DE 19910964 A1 or DE 102008020386 A1, a sidestream of liquid 1,2-dichloroethane is withdrawn, from the downpipe of the reactor, for example, and is cooled via at least one heat exchanger. The cooled 1,2-dichloroethane serves as the motive stream in a liquid jet gas compressor (e.g., an injector nozzle) which draws in gaseous chlorine. Owing to the low temperature of the motive stream and the consequent ready solubility of the chlorine in EDC, the chlorine is already completely dissolved in 1,2-dichloroethane at the exit from the liquid jet gas compressor. This solution is then supplied again to the riser pipe of the reactor by way of a liquid distributor, where it reacts with the dissolved ethylene to form 1,2-dichloroethane.

The liquid-phase reaction regime results in significantly reduced formation of byproducts relative to other processes. In particular, at reaction temperatures of typically 120° C., product purities are achieved for which other processes require substantially lower reaction temperatures in order to suppress unwanted side reactions.

For temperature control, there must be overall removal from the process of heat, which can be used for the heating of heat sinks in other processes. For example, the 1,2-dichloroethane vapor from the top of the reactor may be used for the shell-side heating of the circulation vaporizer of a distillation column or may be fed directly in vapor form into a distillation column for further purification, in each case economizing on the equivalent vapor quantities which would be otherwise needed for column heating.

Similarly, the 1,2-dichloroethane stream drawn off from the reactor for dissolving chlorine may be cooled by shell-side heating, for example, of the circulation vaporizer of a distillation column. A process in which these methods are employed is described for example in EP 1 228 022 B1. Further heat recovery measures may also be realized by means of the vaporous and the liquid 1,2-dichloroethane stream. For example, EP 1 899 287 B1 describes a process in which the heat of reaction from the direct chlorination is used for heating a plant for the concentration of soda lye from a chloralkali-process electrolysis plant.

A further option for the use of the heat of reaction is described by DE 10 2011 014 131 A1, in which the heat of reaction is utilized for drying a wet polymer powder (especially PVC).

A feature common to all of the processes described here is that only part of the sensible heat content of the liquid 1,2-dichloroethane stream can be utilized, as the sensible heat must typically be taken off at above around 95° C.

To be able to dissolve the chlorine reactant in the liquid 1,2-dichloroethane stream, however, the latter must be cooled to about 50 to 60° C. This is normally accomplished by water cooling, and so part of the sensible heat of the 1,2-dichloroethane stream cannot be utilized, owing to the low temperature level. Overall, therefore, in the prior art described, only about 60% of the heat of reaction from the direct chlorination (around 600 kWh/t 1,2-dichloroethane) can be utilized by measures for heat recovery.

Against this background, there is a need for a method which permits more extensive and as far as possible complete utilization of heat produced in the direct chlorination of ethylene. The present invention addresses this need.

### DESCRIPTION OF THE INVENTION

The inventors have ascertained that, surprisingly, extensive utilization of the heat obtained in the direct chlorination of ethylene can be utilized for water desalination. This is a great advantage in particular for sites at which salt water, in the form of seawater for example, is present in large quantities, but hardly any fresh water is available due to precipitation levels. Desalinated water generated accordingly may be used, for example, for a chloralkali process producing the chlorine that is needed for direct chlorination.

The present invention relates accordingly in a first aspect to a method for generation of 1,2-dichloroethane from ethylene and chlorine and for water desalination, by reacting ethylene with chlorine to 1,2-dichloroethane in a first plant part and performing a water desalination in a second plant part, wherein the heat produced in the reaction of ethylene with chlorine is utilized for heating of water in the desalination.

The water desalination preferably comprises a desalination of water having a salt content of 2% to 5% by weight and more particularly 2.8% to 3.8% by weight. Very preferably, the water desalinated has a salt content of about 3.5% by weight—that is, in particular, seawater.

The manner in which the water desalination is performed is not relevantly important for the method of the invention, as long as, as part of the method, the water for purification is heated to a higher temperature, or cooling of the water is to be counteracted by supply of heat. Preferably, however, the method for water desalination is a method in which the water is separated from dissolved salt by vaporization. Cited as a particularly suitable method of this type may be, for example, a multistage flash evaporation (MFS) or a multieffect distillation (MED), of which in particular a multieffect distillation is advantageous. Both of these methods are multistage methods in which the heat fed in can be utilized repeatedly at respectively falling temperature and pressure levels, and they are suitable in particular for utilizing process heat obtained at a low temperature level in order to generate fresh water. Such methods enable utilization even of low-grade process heat, of the kind obtained in the direct chlorination of ethylene, for example, to obtain fresh water from seawater.

Thermal methods for desalination of salt water and more particularly seawater have experienced significant improvements in their efficacy within recent years. For example, WO 2015/154142 A1 describes methods which permit the energy needed for water vaporization to be supplied at a very low temperature level. These methods, also referred to as “boosted multieffect distillation” and “flash-boosted multieffect distillation”, are particularly preferred in the context of the invention described here, as methods for the water desalination which takes place in the second plant part. For a detailed description of these methods, WO 2015/154142 A1 is referenced. The invention, however, is not restricted to the use of a method according to WO 2015/154142 A1.

As mentioned above, a particular advantage is that water generated in a water desalination can be utilized for a chloralkali process and, depending on the technology used for the water desalination, it is possible to generate more water in the water desalination than is needed to operate the chloralkali process. Accordingly, in one preferred embodiment, the method of the invention comprises a third plant part in which a chloralkali process is performed, wherein desalinated water generated in the second plant part is utilized at least fractionally for the chloralkali process. The chlorine generated in the chloralkali process is preferably fed in at least fractionally in the first plant part, where it is reacted with ethylene to 1,2-dichloroethane. The plant part for reaction of ethylene with chlorine is preferably embodied in the method of the invention as a loop reactor, which comprises a riser pipe, an outgassing vessel, and a downpipe.

In a further embodiment, the method of the invention may provide make-up water for polymerization of vinyl chloride to polyvinyl chloride via thermal seawater desalination using heat of reaction from direct chlorination.

A loop reactor may consist, as in the processes of DE 19910964 A1 and DE 102008020386 A1, of a liquid-filled loop which is formed by the riser pipe, the vapor removal zone, and the downpipe. In the riser pipe, ethylene is fed in and chlorine in solution in 1,2-dichloroethane is added, the chlorine having been dissolved in liquid 1,2-dichloroethane beforehand, in an injector, for example. In the case of the illustrative process of DE 19910964 A1 or DE 102008020386 A1, this dichloroethane is diverted from the downpipe out of the reaction mixture and cooled in a cooler to a low temperature in order to facilitate the dissolution of the chlorine. The outgassing vessel may comprise a take-off apparatus for liquid 1,2-dichloroethane and/or a take-off apparatus for gaseous 1,2-dichloroethane (usually and preferably, both are present). The respective supply points and take-off apparatuses may also be implemented multiply, for practical reasons. In the riser pipe of the liquid-filled loop, chlorine and ethylene react with one another to boiling 1,2-dichloroethane, which exits as a vapor in the vapor removal zone together with unreacted starting materials and inert accompanying gas.

Within the method of the invention, preferably, the heat of a 1,2-dichloroethane stream utilized for reaction of ethylene with chlorine and/or heat of condensation from a product from the reaction that is drawn off in vapor form at a reactor top is utilized for heating of water in the desalination. In other words, heat from the 1,2-dichloroethane stream is utilized by routing the 1,2-dichloroethane reaction mixture out of the outgassing vessel or in the region of the downpipe out of the reactor and transferring the heat to a different, preferably liquid, medium by means of a heat exchanger. The 1,2-dichloroethane reaction mixture is subsequently supplied again in the region of the vapor removal zone, in the region of the downpipe, or at a location on the riser pipe ahead of supply points for ethylene. Heat of condensation from a product drawn off in vapour form at a reactor top is likewise advantageously transferred to a preferably liquid heat transfer medium by way of a heat exchanger, with 1,2-dichloroethane condensing from the reaction mixture.

The thermal energy/heat from the 1,2-dichloroethane reaction can be routed directly into the water desalination, by bringing the hot product from the reaction into thermal contact with water to be desalinated, in a heat exchanger (heat transfer without heat transfer medium), or a heat transfer medium can be used, which absorbs the heat energy from the reaction products from 1,2-dichloroethane production (e.g., in a first heat exchanger) and from which the heat energy subsequently transfers to the water to be desalinated (e.g., in a second heat exchanger, which is usually a constituent of the plant part for water desalination). For structural reasons, heat transfer by means of a heat transfer medium is preferred here.

A particularly cost-effective heat transfer medium, suitable by virtue of its high heat capacity, is water, which even enables cooling of the reaction mixture to temperatures of well below 95° C. Accordingly, in one preferred embodiment of the method of the invention, water is utilized as heat transfer medium for the transfer of the heat from the first to the second plant part.

Cooling to temperatures of below 100° C. and more particularly below 80° C. makes it possible to ensure ready solubility of chlorine gas in 1,2-dichloroethane, and so for the method it is preferred if at least part of the 1,2-dichloroethane is cooled, by the transfer of heat to a heat transfer medium, to a temperature in the range from 40 to 90° C., preferably 45 to 85° C., more preferably 50 to 80° C., and more preferably still 50 to 65° C. Advantageously, chlorine gas is subsequently introduced into this part, preferably by way of an injector.

The cooling water which is utilized for cooling the aforesaid 1,2-dichloroethane is preferably heated to not more than 95° C., more preferably 90° C. and more preferably still not more than 85° C.

In a further aspect, the present invention relates to a use of heat from a method for generation of 1,2-dichloroethane from ethylene and chlorine for heating of water to be treated in a water desalination, wherein the heat is transferred from 1,2-dichloroethane to the water by means of a transfer apparatus. For preferred configurations of this use, reference may be made to the observations above regarding the corresponding method.

In yet a further aspect, the present invention relates to an integrated plant for production of 1,2-dichloroethane and for water desalination, wherein the integrated plant comprises a first plant part with a reactor for reaction of chlorine with ethylene to 1,2-dichloroethane, a second plant part for water desalination, and an apparatus for transfer of heat energy between the two plant parts. The plant is preferably adapted/configured for performance of a method as described above.

The plant is an integrated plant, meaning that the first and second plant parts are spatially adjacent or arranged in spatial vicinity and there are one or more conduits present allowing heat energy to be transmitted from the first plant part to the second plant part.

In one preferred embodiment, the plant additionally comprises a plant part for the chloralkali process, wherein the plant part for water desalination is fluidly connected to the plant part for the chloralkali process, to allow desalinated water from the water desalination to be supplied to the chloralkali process. “Fluidly connected” here means that there is a conduit present communicating on the one hand with the product side of the water desalination and on the other hand with the starting-material side of the chloralkali process. In one particularly preferred embodiment, the plant additionally has a conduit allowing chlorine produced in the chloralkali process (i.e., chlorine gas, Cl2) to be transferred into the plant part for production of 1,2-dichloroethane.

In another preferred embodiment, the plant additionally comprises a plant part for polymerization of vinyl chloride, wherein the plant part for water desalination is fluidly connected to the plant part for polymerization of vinyl chloride, to allow desalinated water from the water desalination to be supplied to the polymerization of vinyl chloride. “Fluidly connected” here means that there is a conduit present communicating on the one hand with the product side of the water desalination and on the other hand with the starting-material side of the polymerization of vinyl chloride. In the polymerization of vinyl chloride, the water is used advantageously as make-up water.

In the plant described, the plant part for water desalination is expediently embodied as a multistage flash evaporation, multieffect distillation, boosted multieffect distillation or flash-boosted multieffect distillation. Alternatively and preferably additionally to this, the reactor, in the plant part for reaction of chlorine with ethylene to 1,2-dichloroethane, is embodied as a loop reactor. It is preferable, furthermore, if the plant comprises one or more heat exchangers which communicate by way of conduits with the headspace of an outgassing vessel of the loop reactor and with the outgassing vessel or the downpipe in such a way that with the reactor in operation, liquid material can be passed to the heat exchanger. In an especially preferred embodiment, the other side of the heat exchanger communicates with a circulation system for heat transfer fluid, which is in turn coupled thermally with the plant part for water desalination.

In the text below, the present invention and also embodiments thereof are illustrated in more detail with figures:

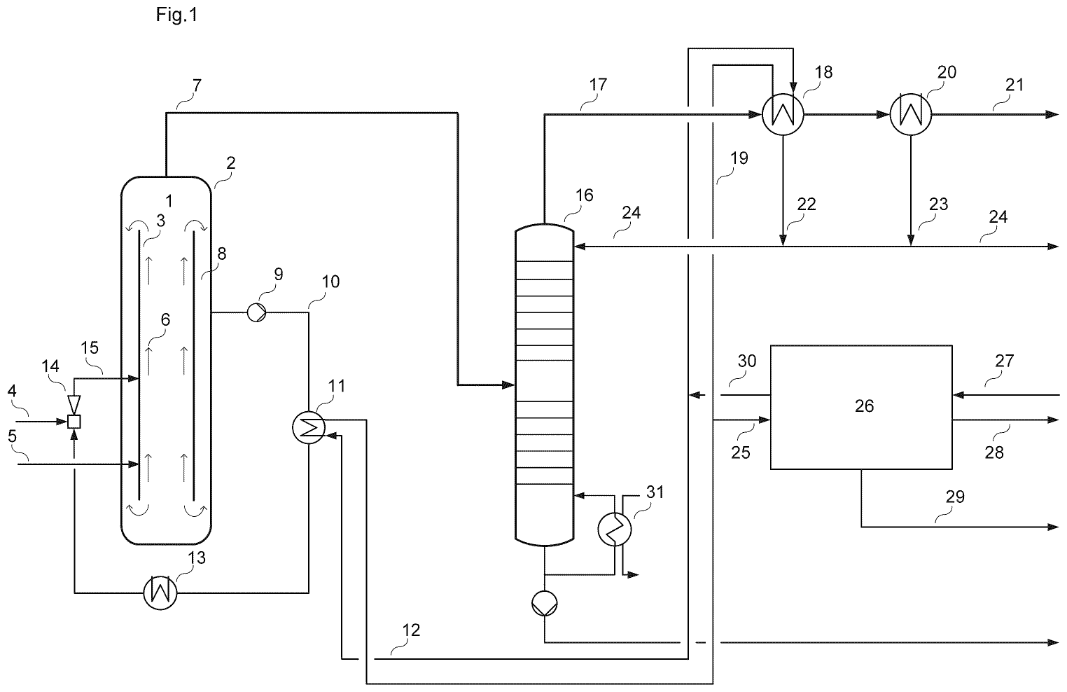

FIG. 1 shows an illustrative embodiment of a plant of the invention for direct chlorination of ethylene with a thermal seawater desalination plant.

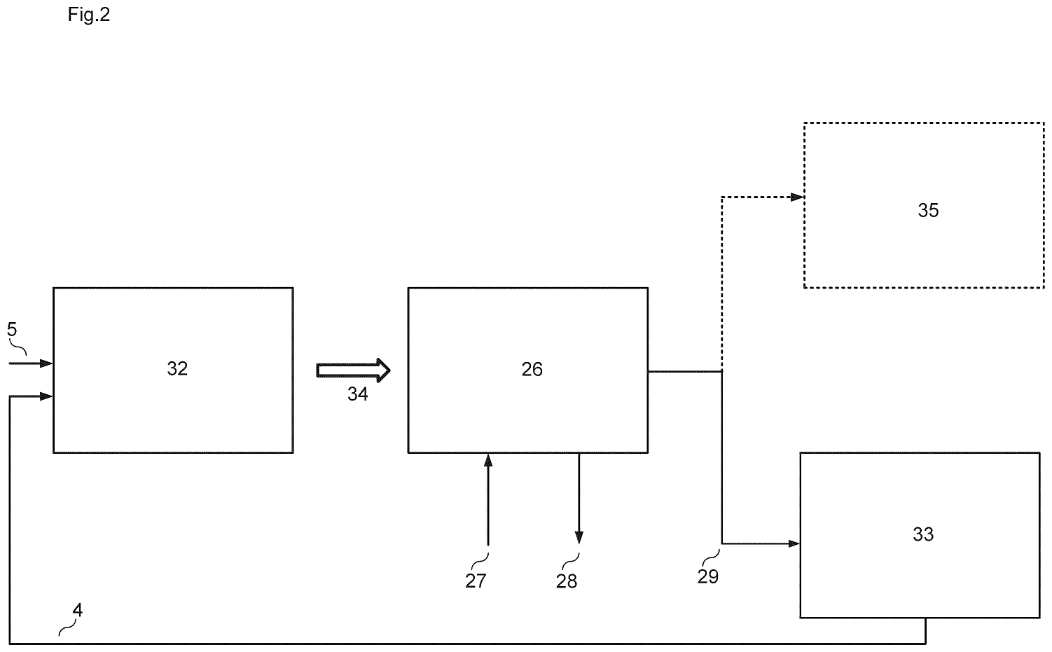

FIG. 2 shows an illustrative, schematic embodiment of a plant configuration of the invention.

FIG. 1 is elucidated more closely as follows:

In a loop reactor (1), consisting of a reaction vessel (2) and an internal riser pipe (3), chlorine (4) and ethylene (5) are reacted to EDC in a circulating, liquid EDC stream (6). In the upper part of the reactor, the reaction mixture boils and the product (7) is taken off in vapor form from the reactor. Ethylene is added in the lower part of the riser pipe (3) by way of a manifold apparatus (not represented) and dissolves in the circulating EDC stream.

An EDC substream (10) is withdrawn from the annular gap of the reactor (8) via an EDC circulation pump (9) and cooled in a first circulation cooler (11), with initial heating of a first hot water substream (12). In a second EDC circulation cooler (13), the EDC substream is cooled further, where appropriate, to the temperature required for use in the reaction, and is used in a jet pump (14) for drawing in the chlorine (4) and dissolving it. The chlorine-containing EDC circulation stream (15) is then added by way of a manifold apparatus (not represented) in the riser pipe (3) to the circulating EDC stream, which already contains dissolved ethylene. The direct chlorination reaction then takes place in the liquid phase.

In the upper part of the riser pipe (3), as a result of a decrease in the hydrostatic pressure, the reaction mixture begins to boil and undergoes partial vaporization. EDC in vapor form (7) is taken off at the top of the reactor and fed into a distillation column (16) for removal of relatively high-boiling byproducts. The pure product (17) is taken off at the top of the columns and predominantly condensed via a top condenser (18), with initial heating of a second hot water substream (19). Following condensation of further EDC in at least one secondary condenser (20), the remaining offgas stream (21) is sent to the plant limits for further processing. The condensed product EDC streams (22), (23) are fed partially as return flow (24) to the column. The remaining quantity of EDC is sent as a product to the plant limits. The external quantity of heat required for the distillation is supplied by the circulation vaporizer (31) at the column bottom.

The combined, preheated water streams (12), (19) are supplied as initial hot water fraction (25) to a multistage plant for thermal seawater desalination (26). Sea water (27) is fed into the desalination plant, while a concentrated sea water stream (28) is passed back to the sea. Fresh water (29) is supplied for further use inside or outside the plant complex. The hot water return stream (30) is divided and supplied for heating the heat exchangers (11) and (18) again.

FIG. 2 is elucidated more closely as follows:

The materials flows and heat flows stated in the example relate to a dichloroethane capacity of around 327 kt/a 1,2-dichloroethane, which in a plant referred to as a balanced plant for production of vinyl chloride/polyvinyl chloride would correspond to a vinyl chloride/polyvinyl chloride capacity of 400 kt/a. Volume flows are stated only as and where necessary to elucidate the invention.

In a direct chlorination plant (32), chlorine (4) from a chloralkali process plant (33) and ethylene (5) are reacted with one another to 1,2-dichloroethane (not represented). The heat of reaction corresponds to a thermal output of around 25 MW.

Around 18 MW of thermal power (34) are coupled out of the direct chlorination (32) and used for heating a plant for thermal seawater desalination (26). This corresponds to a recovery rate of around 72%. For this, seawater (27) is supplied to the seawater desalination plant, and seawater (28) concentrated by vaporization is passed back to the plant limits again.

Around 92 t/h (around 2200 t/d) desalinated water (29) can be obtained. The chloralkali process plant has a desalinated water demand of around 2100 t/d. The water demand of the chloralkali process plant can therefore be covered entirely by thermal desalination of seawater using the heat of reaction from the direct chlorination.

Alternatively (10), the desalinated water can be used as make-up water in a plant for production of polyvinyl chloride (35). The water demand of the polyvinyl chloride plant is around 2760 t/d and can be covered to an extent of around 76%.

The invention is not, however, confined to the examples according to FIGS. 1 and 2. In particular, a higher fraction of the heat of reaction from the direct chlorination can be recovered and/or more desalinated water produced, through increases in the level of apparatus deployed.

### LIST OF REFERENCE SIGNS

1 loop reactor
2 reaction vessel
3 riser pipe
4 chlorine
5 ethylene
6 circulating EDC stream
7 product EDC, in vapor form
8 annular gap
9 EDC circulation pump
10 EDC substream
11 EDC circulation cooler I
12 hot water substream I
13 EDC circulation cooler II
14 jet pump
15 chlorine-containing EDC circulation stream
16 high-boilers:
17 product EDC, in vapor form
18 top condenser
19 hot water substream II
20 secondary condenser
21 offgas
22 product EDC, in liquid form
23 product EDC, in liquid form
24 return flow
25 initial hot water fraction
26 thermal seawater desalination plant
27 sea water
28 sea water, concentrate
29 fresh water
30 hot water return flow
31 circulation vaporizer
32 direct chlorination plant
33 chloralkali process plant
34 heat of reaction from direct chlorination
35 PVC plant (optional)

1-14. (canceled)

15. A method for generation of 1,2-dichloroethane from ethylene and chlorine and for water desalination, comprising:
reacting ethylene with chlorine to 1,2-dichloroethane in a first plant part; and
performing a water desalination in a second plant part,
wherein the heat produced in the reaction of ethylene with chlorine is utilized for heating of water in the desalination,
wherein the water desalination is performed as multistage flash evaporation or as multieffect distillation,
wherein water is utilized as a heat transfer medium for transfer of the heat from the first to the second plant part,
wherein the water for cooling of the 1,2-dichloroethane is heated to not more than 95° C.

16. The method as claimed in claim 15, wherein the water desalination is performed as seawater desalination.

17. The method as claimed in claim 15, wherein the water produced in the reaction of ethylene with chlorine is utilized for heating of a thermal process for water desalination via boosted multieffect distillation or flash-boosted multieffect distillation.

18. The method as claimed in claim 15, further comprising a third plant part, in which a chloralkali process is performed and where desalinated water generated in the second plant part is utilized at least fractionally for the chloralkali process.

19. The method as claimed in claim 15, wherein the reaction of ethylene with chlorine to 1,2-dichloroethane is performed in a loop reactor.

20. The method as claimed in claim 15, wherein the heat of a 1,2-dichloroethane stream utilized for reaction of ethylene with chlorine and/or heat of condensation from a product from the reaction that is drawn off in vapor form at a reactor top is utilized for heating of water in the desalination.

21. The method as claimed in claim 20, wherein at least part of the 1,2-dichloroethane is cooled, by transfer of heat to a heat transfer medium, to a temperature in the range from 40 to 90° C.

22. The method as claimed in claim 20, wherein at least part of the 1,2-dichloroethane is cooled, by transfer of heat to a heat transfer medium, to a temperature in the range from 50 to 65° C.

23. A method, comprising:
using heat from generation of 1,2-dichloroethane from ethylene and chlorine for heating of water to be treated in a water desalination,
wherein the heat is transferred from 1,2-dichloroethane to the water by means of a transfer apparatus,
wherein the heat produced in the reaction of ethylene with chlorine is utilized for heating of a thermal process for water desalination via boosted multieffect distillation or flash-boosted multieffect distillation,
wherein water is utilized as heat transfer medium for transfer of the heat from the 1,2-dichloroethane to the water,
wherein the water for cooling of the 1,2-dichloroethane is heated to not more than 95° C.

24. A plant for production of 1,2-dichloroethane and for water desalination, comprising:
a first plant part with a reactor for reaction of chlorine with ethylene to 1,2-dichloroethane,
a second plant part for water desalination, and
an apparatus for transfer of heat energy between the two plant parts,
wherein the plant part for water desalination is designed as a boosted multieffect distillation or flash-boosted multieffect distillation and the plant is configured such that transfer of heat energy between the first and second plant part is possible via water as heat transfer medium, and the second plant part for water desalination is configured for operation with water as heat transfer medium at a temperature of not more than 95° C.

25. The plant as claimed in claim 24, wherein the plant additionally comprises a plant part for a chloralkali process, and wherein the plant part for water desalination is fluidly connected to the plant part for the chloralkali process, to allow desalinated water from the water desalination to be supplied to the chloralkali process.

26. The plant as claimed in claim 24, wherein the plant part for water desalination is embodied as a multistage flash evaporation, multieffect distillation, boosted multieffect distillation or flash-boosted multieffect distillation.

27. The plant as claimed in claim 24, wherein the reactor, in the plant part for reaction of chlorine with ethylene to 1,2-dichloroethane, is embodied as a loop reactor.
